(19) United States
(12) Patent Application Publication
(10) Pub. No.: US 2025/0072986 A1
(43) Pub. Date: Mar. 6, 2025

(54) ORIENTING IMAGE FOR ROBOTIC SURGERY

(71) Applicant: ForSight Robotics Ltd., Yokneam Illit (IL)

(72) Inventors: Tal KORMAN, Tel Aviv (IL); Ori BEN ZEEV, Ramat Hasharon (IL); Yoav GOLAN, Tel-Aviv (IL)

(21) Appl. No.: 18/822,932

(22) Filed: Sep. 3, 2024

Related U.S. Application Data
(60) Provisional application No. 63/536,772, filed on Sep. 6, 2023.

(51) Int. Cl. A61B 34/37; A61B 17/00; A61B 34/00; A61B 90/00; A61F 9/007

(52) U.S. Cl. CPC A61B 34/37; A61B 34/77; A61B 90/361; A61B 2017/00216; A61B 2090/367; A61B 2090/373; A61F 9/007

(57) ABSTRACT

A system for performing robotic surgery at a surgical site on a patient includes a control tool configured for manipulation by an operator, one or more displays, and a processor configured to cause a robotic arm, which holds a surgical tool, to manipulate the surgical tool correspondingly to the manipulation of the control tool, such that the operator controls the surgical tool via the control tool, to cause the displays to display a real-time video of the surgical site, in which the surgical site is magnified, to the operator while the operator controls the surgical tool, and to cause the displays to display at least one three-dimensional (3D) image of the surgical site, in which the surgical site is shown with less magnification relative to the video. Other embodiments are also described.

### CROSS-REFERENCE TO RELATED APPLICATIONS

The present application claims the benefit of U.S. Provisional Application No. 63/536,772, entitled “Orienting image for robotic surgery,” filed Sep. 6, 2023, whose disclosure is incorporated herein by reference.

### FIELD OF THE INVENTION

The present invention is related to the field of robotic surgery, such as robotic microsurgery.

### BACKGROUND OF THE INVENTION

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and the face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye, in order to further anesthetize the eye. Following this step, a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incision, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. Further typically, a separate tool is used to perform suction during the phacoemulsification. When the phacoemulsification is complete, the remaining lens cortex (i.e., the outer layer of the lens) material is aspirated from the capsule. During the phacoemulsification and the aspiration, aspirated fluids are typically replaced with irrigation of a balanced salt solution, in order to maintain fluid pressure in the anterior chamber. In some cases, if deemed to be necessary, the capsule is polished. Subsequently, the intraocular lens (IOL) is inserted into the capsule. The IOL is typically foldable and is inserted in a folded configuration, before unfolding inside the capsule. At this stage, the viscoelastic is removed, typically using the suction device that was previously used to aspirate fluids from the capsule. If necessary, the incision(s) is sealed by elevating the pressure inside the bulbus oculi (i.e., the globe of the eye), causing the internal tissue to be pressed against the external tissue of the incision, such as to force closed the incision.

### SUMMARY OF THE INVENTION

In some types of robotic surgery, one or more robotic arms manipulate respective surgical tools under the control of an operator. In particular, the operator manipulates one or more control tools, and a computer processor drives the robotic arms to manipulate the surgical tools in a corresponding manner, so as to perform a surgical procedure on a patient. While controlling the robotic arms, the operator views a real-time video of the surgical site, which is displayed on a primary display typically positioned ahead of the operator, e.g., at the operator's eye level.

Typically, the field of view (FOV) of the camera that acquires the real-time video is relatively small, so as to facilitate a magnified view of the surgical site. Alternatively or additionally, the orientation of the camera may change during the procedure. Due to the small FOV of the camera, the magnification of the surgical site, and/or the changing orientation of the camera, the operator may become disoriented. For example, the operator may lose his awareness of where the robotic arms are positioned relative to the body of the patient (or equivalently, where the operator's hands would be positioned if the procedure were being performed frontally, without a robotic intermediary), and/or how this position changes with movement of the control tools.

Embodiments of the present invention therefore provide a system configured to address the aforementioned challenge. The system, which is for performing robotic surgery at a surgical site on a patient, includes a control tool, which is configured for manipulation by an operator, one or more displays, and a processor. The processor is configured to cause a robotic arm, which holds a surgical tool, to manipulate the surgical tool correspondingly to the manipulation of the control tool, such that the operator controls the surgical tool via the control tool. The processor is also configured to cause the displays to display a real-time video of the surgical site, in which the surgical site is magnified, to the operator while the operator controls the surgical tool. In addition, the processor is configured to cause the displays to display at least one three-dimensional (3D) image of the surgical site, in which the surgical site is shown with less magnification relative to the video (e.g., without any magnification). Thus, advantageously, the operator may reorient himself by viewing the 3D image.

Typically, to facilitate the aforementioned reorientation, the field of view (FOV) shown in the 3D image is larger than the FOV shown in the video. For example, for cases in which the surgical site includes part of an eye of the patient (and hence, the real-time video shows a magnified view of the eye), the 3D image may show the face of the patient, such that the operator may better understand where the surgical tools are positioned relative to the face.

In some embodiments, the 3D image is displayed on a 3D display. In other embodiments, the 3D image is displayed on an extended-reality display, such as an augmented-reality display. For example, the system may include a device wearable over the eyes of the operator and including the extended-reality display and an orientation sensor configured to communicate, to the processor, a signal indicating the orientation of the device. The processor is configured to cause the extended-reality display to display the 3D image in response to the signal indicating that the operator is looking toward the vicinity of the control tool, rather than toward the primary display.

There is therefore provided, in accordance with some embodiments of the present invention, a system for performing robotic surgery at a surgical site on a patient. The system includes a control tool, configured for manipulation by an operator, one or more displays, and a processor. The processor is configured to cause a robotic arm, which holds a surgical tool, to manipulate the surgical tool correspondingly to the manipulation of the control tool, such that the operator controls the surgical tool via the control tool. The processor is further configured to cause the displays to display a real-time video of the surgical site, in which the surgical site is magnified, to the operator while the operator controls the surgical tool. The processor is further configured to cause the displays to display at least one three-dimensional (3D) image of the surgical site, in which the surgical site is shown with less magnification relative to the video.

In some embodiments, a spatial relationship between the control tool and the surgical site as shown in the 3D image corresponds to a spatial relationship between the surgical tool and the surgical site.

In some embodiments, the surgical site is shown in the 3D image without any magnification.

In some embodiments, a size of the surgical site as shown in the 3D image corresponds to an actual size of the surgical site.

In some embodiments,
the processor is configured to scale movement of the surgical tool, relative to movement of the control tool, by a scale factor, and
a size of the surgical site as shown in the 3D image is scaled, relative to an actual size of the surgical site, by an inverse of the scale factor.

In some embodiments, the real-time video is 3D.

In some embodiments, the processor is further configured to mark a control zone in the 3D image, the control zone being a predefined volume in which the surgical tool is controllable by the operator.

In some embodiments, the at least one 3D image includes a real-time 3D video.

In some embodiments, the 3D image shows the surgical tool.

In some embodiments, a field of view (FOV) shown in the 3D image is larger than an FOV shown in the video.

In some embodiments, the surgical site includes part of an eye of the patient, and the 3D image shows a face of the patient.

In some embodiments, the processor is further configured to mark, in the 3D image, the FOV shown in the video.

In some embodiments,
the displays include a 3D display, and
the processor is configured to cause the 3D display to display the 3D image.

In some embodiments, the system further includes a sensor configured to communicate, to the processor, a signal indicating a gaze direction of the operator,
and the processor is further configured to lock the control tool responsively to the signal indicating that the operator is looking toward the 3D display.

In some embodiments, the sensor includes an imaging sensor.

In some embodiments, the 3D display is configured to use the imaging sensor to track eyes of the operator as the operator looks toward the 3D display, and to display the 3D image responsively to tracking the eyes of the operator.

In some embodiments,
the sensor includes an orientation sensor, and
the system further includes a device wearable over eyes of the operator and including:
   lenses configured for viewing the 3D image via the lenses; and the orientation sensor.

In some embodiments, the 3D display is positioned underneath the control tool.

In some embodiments, the 3D display is tilted such that a top of the 3D display, which is positioned to be farther from the operator, is higher than a bottom of the 3D display, which is positioned to be closer to the operator.

In some embodiments,
the displays include an extended-reality display, and
the processor is configured to cause the extended-reality display to display the 3D image.

In some embodiments, the system further includes a device wearable over eyes of the operator and including:
the extended-reality display; and
an orientation sensor configured to communicate, to the processor, a signal indicating an orientation of the device,
and the processor is configured to cause the extended-reality display to display the 3D image in response to the signal indicating that the operator is looking toward a vicinity of the control tool.

In some embodiments, the processor is further configured to lock the control tool responsively to the signal indicating that the operator is looking toward the vicinity of the control tool.

In some embodiments,
the extended-reality display is an augmented-reality display, and
the processor is further configured to augment the control tool with one or more portions of the surgical tool in the 3D image.

There is further provided, in accordance with some embodiments of the present invention, a method for performing robotic surgery at a surgical site on a patient. The method includes causing a robotic arm, which holds a surgical tool, to manipulate the surgical tool correspondingly to manipulation of a control tool by an operator, such that the operator controls the surgical tool via the control tool. The method further includes causing one or more displays to display a real-time video of the surgical site, in which the surgical site is magnified, to the operator while the operator controls the surgical tool. The method further includes causing the displays to display at least one three-dimensional (3D) image of the surgical site, in which the surgical site is shown with less magnification relative to the video.

There is further provided, in accordance with some embodiments of the present invention, a computer software product including a tangible non-transitory computer-readable medium in which program instructions are stored. The instructions, when read by a processor, cause the processor to perform robotic surgery at a surgical site on a patient by causing a robotic arm, which holds a surgical tool, to manipulate the surgical tool correspondingly to manipulation of a control tool by an operator, such that the operator controls the surgical tool via the control tool, causing one or more displays to display a real-time video of the surgical site, in which the surgical site is magnified, to the operator while the operator controls the surgical tool, and causing the displays to display at least one three-dimensional (3D) image of the surgical site, in which the surgical site is shown with less magnification relative to the video.

The present invention will be more fully understood from the following detailed description of embodiments thereof, taken together with the drawings, in which:

### BRIEF DESCRIPTION OF THE DRAWINGS

FIG. 1 is a schematic illustration of a system for performing robotic surgery at a surgical site on a patient, in accordance with some embodiments of the present invention;

FIG. 2 is a schematic illustration of an augmented-reality display of an orienting image, in accordance with some embodiments of the present invention;

FIG. 3 is a schematic illustration of a surgical workspace, in accordance with some embodiments of the present invention;

FIG. 4 is a schematic illustration of an operator using the surgical workspace shown in FIG. 3, in accordance with some embodiments of the present invention; and

FIG. 5 is a schematic illustration of an alternate surgical workspace, in accordance with some embodiments of the present invention.

### DETAILED DESCRIPTION

### Overview

In some types of robotic surgery, one or more robotic arms manipulate respective surgical tools under the control of an operator. In particular, the operator manipulates one or more control tools, and a computer processor drives the robotic arms to manipulate the surgical tools in a corresponding manner, so as to perform a surgical procedure on a patient. While controlling the robotic arms, the operator views a real-time video of the surgical site, which is displayed on a primary display typically positioned ahead of the operator, e.g., at the operator's eye level.

Typically, the field of view (FOV) of the camera that acquires the real-time video is relatively small, so as to facilitate a magnified view of the surgical site. Alternatively or additionally, the orientation of the camera may change during the procedure. Due to the small FOV of the camera, the magnification of the surgical site, and/or the changing orientation of the camera, the operator may become disoriented. For example, the operator may lose his awareness of where the robotic arms are positioned relative to the body of the patient (or equivalently, where the operator's hands would be positioned if the procedure were being performed frontally, without a robotic intermediary), and/or how this position changes with movement of the control tools.

To address this problem, embodiments of the present invention provide a secondary display, which supplements the primary display. The processor drives this secondary display to display a three-dimensional (3D) image, referred to herein as an “orienting image,” which shows the surgical site with less magnification, typically along with surrounding portions of the patient's anatomy. Thus, the operator may become reoriented by viewing the orienting image.

In some embodiments, the orienting image is displayed such that the spatial relationship between the controls tool and the surgical site as shown in the image corresponds to the spatial relationship between the surgical tools and the surgical site. For example, for an ophthalmic microsurgical procedure, a robotic arm may grasp a surgical tool such that the proximal end of the tool is positioned over the patient's nose and the distal end of the tool is positioned at the patient's eye. In such a case, the orienting image may show the face of the patient positioned such that the proximal end of the operator's control tool is over the patient's nose and the distal end of the control tool is at the patient's eye. The operator may thus feel as if he were performing the procedure frontally, without a robotic intermediary.

In some embodiments, the size of the surgical site as shown in the image corresponds to the actual size of the surgical site, i.e., the scale of the image, relative to the real world, is approximately one (e.g., between 0.8 and 1.2, such as between 0.9 and 1.1). This correspondence in size may further facilitate maintaining an awareness of where the robotic arms are positioned relative to the body of the patient.

In other embodiments, the size of the surgical site is scaled, relative to its actual size, inversely to the scaling of movement of the surgical tools relative to movement of the control tools. For example, if a movement of 2 mm by a control tool causes a 1 mm movement of the corresponding surgical tool, the size of the surgical site in the image may be double its actual size. This scaling may further facilitate maintaining an awareness of how the positions of the robotic arms change with movement of the control tools. (Optionally, the operator may toggle between a first display mode, in which the surgical site is displayed at its actual size, and a second display mode, in which the surgical site is scaled.)

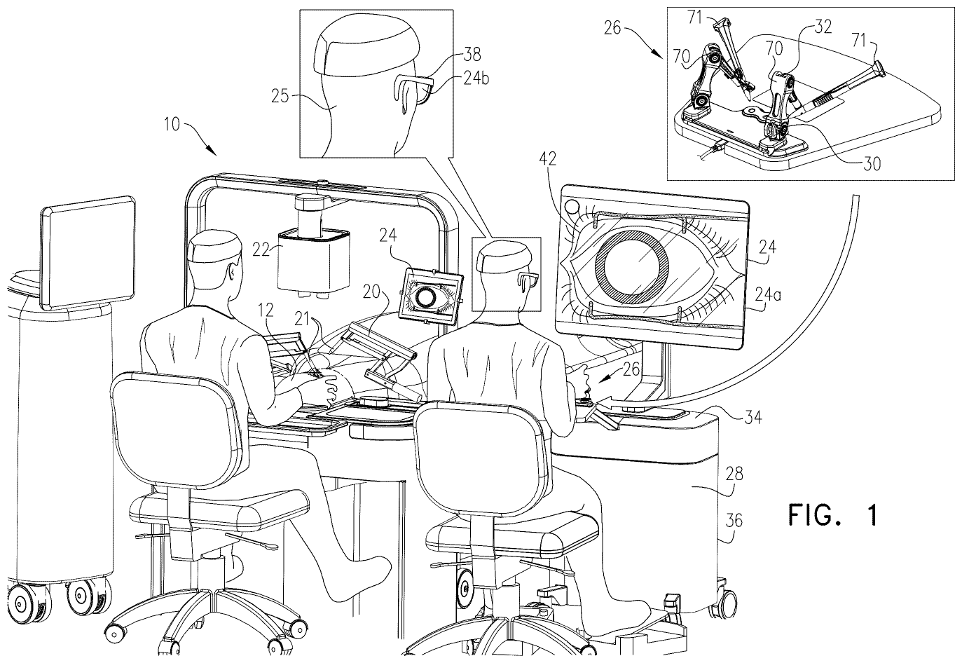

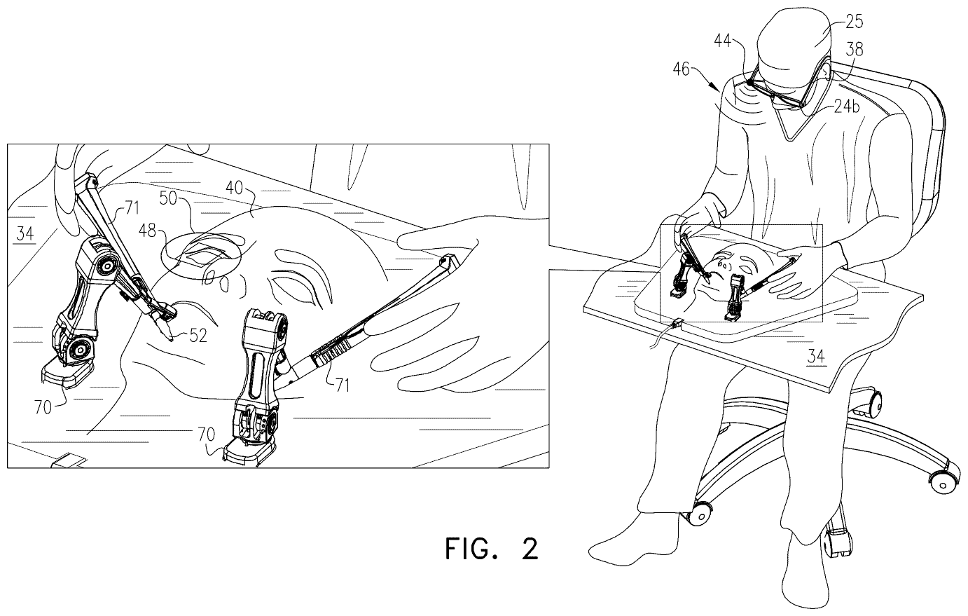

In some embodiments, as shown in FIG. 2, the secondary display is an extended-reality display 24b, i.e., a display configured for the display of virtual and/or augmented reality. Typically, such a display belongs to a device worn over the operator's eyes. The device comprises an orientation sensor 44, which tracks the orientation of the operator's head, and the computer processor 28 (FIG. 1) controls the extended-reality display responsively to this tracking. Thus, while the operator looks (e.g., looks ahead) toward the primary display, the extended-reality display does not modify the view of the operator. In contrast, while the operator looks (e.g., looks down) toward the control tools, the extended-reality display augments or replaces the view of the operator with the orienting image.

Thus, for example, the operator may experience a (real-world) view of the control tools augmented with a (virtual) view of the face of the patient. Optionally, the control tools may be augmented with portions of the surgical tools, such that it appears, to the operator, as if the operator were holding the surgical tools.

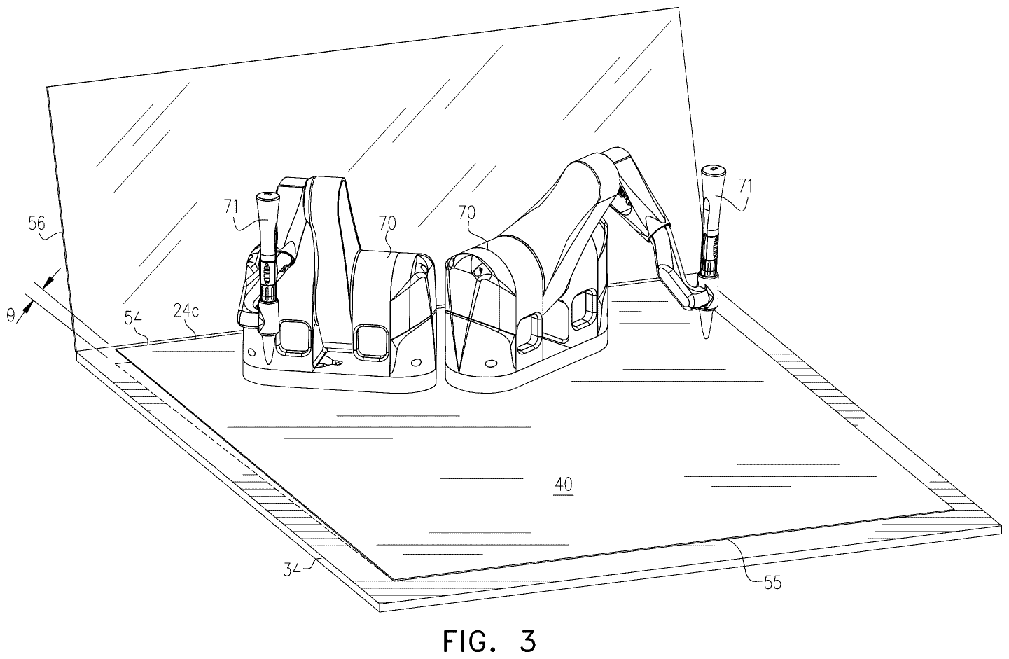

In other embodiments, as shown in FIGS. 3-5, the secondary display is a 3D display 24c (i.e., a display configured for the display of 3D images). In some such embodiments, the 3D display is positioned underneath the control tools, so as to facilitate giving the appearance of performing the procedure frontally.

### System Description

Reference is initially made to FIG. 1, which is a schematic illustration of a system 10 for performing robotic surgery at a surgical site on a patient 12, in accordance with some embodiments of the present invention.

System 10 comprises one or more (e.g., two) robotic arms 20 configured to hold respective surgical tools 21. Surgical tools 21 may include, for example, a blade, tweezers, a phacoemulsification tool, or a fluid-injection tool.

System 10 further comprises a control-component unit 26 and a computer processor 28. An operator 25, such as a physician or other healthcare professional, provides inputs to processor 28 via control-component unit 26. In response to these inputs, the processor communicates corresponding outputs to robotic arms 20, thereby moving the robotic arms (and hence surgical tools 21) in accordance with the inputs.

In particular, control-component unit 26 comprises one or more (e.g., two) control components 70, each of which corresponds to a different respective robotic arm 20. Each control component 70 comprises a control tool 71, which operator 25 holds and manipulates as if control tool 71 were the surgical tool 21 held by the corresponding robotic arm. (Typically, control tool 71 is generically-shaped, such that the size and shape of the control tool do not exactly match the size and shape of the corresponding surgical tool; for example, the control tool may comprise a stylus.) In response to the manipulation of each control tool, the processor causes the corresponding robotic arm 20 to manipulate the surgical tool 21 correspondingly to the manipulation of the control tool, such that the operator controls the surgical tool via the control tool.

For example, operator 25 may control the pose (i.e., the position and orientation) of each surgical tool by appropriate manipulation of the corresponding control tool. In particular, processor 28 may move each surgical tool such that the position of a predetermined portion (e.g., the distal tip) of the surgical tool tracks the position of a predetermined portion of the corresponding control tool, and the orientation of the surgical tool matches that of the control tool. In addition, the operator may control the operations performed by each surgical tool by appropriate manipulation of the corresponding control tool. Such operations may include, for example, a tweezing operation, an application of suction, or an injection of fluid.

In some embodiments, the processor is configured to scale movement of each surgical tool, relative to movement of the corresponding control tool, by a scale factor. Typically, this scale factor is less than one, i.e., for every movement of 1 mm by the control tool, the processor moves the surgical tool by 1/X mm, where X>1.

Typically, each control component 70 comprises a control arm 30 coupled to control tool 71. Control arm 30 comprises multiple joints 32 providing multiple degrees of freedom for the movement of control tool 71, along with respective sensors for joints 32. The sensors are configured to communicate, to processor 28, signals indicative of any changes in the orientations of the joints (and thus, any movement of control tool 71), such that the processor may cause the corresponding robotic arm 20 to move surgical tool 21 in a corresponding manner.

In addition to control components 70, control-component unit 26 may comprise one or more other input interfaces (e.g., buttons). Alternatively or additionally, system 10 may comprise one or more separate input interfaces (e.g., a foot pedal). Such input interfaces may be used, by operator 25, to control the operations performed by each surgical tool.

System 10 further comprises an imaging system 22 comprising, for example, a wide-angle camera, a microscope camera, a light detection and ranging (LiDAR) camera, and/or any other suitable imaging device. System 10 further comprises one or more displays 24. In some embodiments, at least one of the cameras belonging to imaging system 22 is stereoscopic, and at least one of displays 24 is a 3D display.

Imaging system 22 images the surgical site and communicates the images to processor 28. In response to receiving the images, the processor causes displays 24 to display the images. In some embodiments, the processor also processes the images so as to track the pose of each surgical tool. (For example, each tool may comprise a marker, and the processor may compute the pose based on the pose of the marker in the image.) In other embodiments, the pose of each surgical tool is tracked using other techniques, such as electromagnetic tracking.

Typically, displays 24 comprise a primary display 24a and a secondary display, which is further described below. Due to the need for real-time visual feedback while performing the procedure, processor 28 causes primary display 24a to display a real-time video (e.g., a real-time 3D video) of the surgical site, also referred to below as a “main video,” to the operator. In other words, imaging system 22 acquires images of the surgical site in rapid succession (e.g., at a frame rate of at least 60 frames per second), and processor 28 causes primary display 24a to display these images in real-time, e.g., such that the delay between the acquisition of each image and the display of the image is less than 80 ms. (In general, when referring to the frames of a video, the present description may use the terms “image” and “frame” interchangeably.)

Typically, the surgical site is magnified in the real-time video. For example, system 10 may be used to perform microsurgery, and primary display 24a may display a real-time video of the surgical site acquired by a microscope camera. As a specific example, system 10 may be used for ophthalmic microsurgery (e.g., cataract surgery), and primary display 24a may display a real-time video of the eye 42 of patient 12 at which the surgical site is located.

Typically, primary display 24a is positioned, relative to control tools 71, such that, while the operator uses the control tools, the operator looks ahead at the primary display. For example, the primary display may sit on, or be mounted to, a surface in front of operator 25. Typically, primary display 24a is vertically-oriented.

For example, operator 25 may sit or stand at a work surface 34, such as the top surface of a console 36 (which, optionally, may contain processor 28). Primary display 24a may sit on surface 34, and control-component unit 26 may also sit on surface 34, between primary display 24a and operator 25.

In some embodiments, the secondary display is an extended-reality display 24b. For example, as further described below with reference to FIG. 2, system 10 may comprise a device 38, such as glasses or a visor coupled to a helmet, wearable over the eyes of operator 25 and comprising extended-reality display 24b. In other embodiments, as further described below with reference to FIGS. 3-5, the secondary display is a 3D display, which may be positioned underneath the control tools.

Processor 28 is configured to cause the secondary display to display at least one 3D image of the surgical site, referred to herein as an “orienting image.” In some embodiments, the 3D image is acquired, in its entirety, by imaging system 22; for example, the imaging system may comprise a wide-angle stereoscopic camera configured to acquire the 3D image. In other embodiments, the processor generates the 3D image, or at least one or more portions thereof, based on one or more of the original images acquired by imaging system 22. For example, the processor may generate the 3D image by patching together portions of the original images and/or altering an original image, e.g., by changing the viewing perspective so as to correspond to the perspective of a surgeon performing the procedure frontally.

In some embodiments, the processor displays the orienting image such that the spatial relationship between each control tool 71 and the surgical site as shown in the image corresponds to the spatial relationship between the corresponding surgical tool 21 and the surgical site. In other words, the orienting image is displayed such that the pose of the control tool, relative to the surgical site as displayed in the image, corresponds to the pose of the surgical tool relative to the surgical site in the real world. Prior to the procedure, the processor registers the coordinate system of robotic arms 20 with the coordinate system of imaging system 22, such that the processor may continually track the spatial relationship between the surgical tools and the surgical site, and hence, drive the secondary display to display the orienting image with the same spatial relationship.

Typically, the field of view (FOV) shown in the orienting image is larger than the FOV shown in the main video. For example (e.g., when performing robotic ophthalmic microsurgery), the surgical site may include part of eye 42, such that operator 25 sees only the eye in the main video. In contrast, the orienting image may show the face of the patient, such that the operator can better understand where the surgical tools are positioned relative to the face.

To facilitate the larger FOV and/or to otherwise facilitate orienting the operator, the surgical site is shown in the orienting image with less magnification, relative to the video. For example, the surgical site may be shown without any magnification. Alternatively, the size of the surgical site as shown in the 3D image may be scaled, relative to the actual size of the surgical site, by the inverse of the aforementioned scale factor. For example, the size of the surgical site as shown in the 3D image may be a multiple X of the actual size of the surgical site, where X is as defined above.

In some embodiments, the 3D image shows one or more of the surgical tools and, optionally, the robotic arms that hold these tools. Typically, the spatial relationship between the surgical tools and the surgical site is preserved in the 3D image.

In some embodiments, a single, static orienting image is displayed throughout the procedure. In other embodiments, instead of a static image, a 3D video, e.g., having a frame rate of at least 60 frames per second, is displayed in real-time, e.g., such that, for each displayed frame, the delay between the acquisition of the frame and the display of the frame is less than 80 ms. Thus, advantageously, any movement of the patient during the procedure may be accounted for. (Notwithstanding the above, for ease of description, the term “orienting image” is used below to refer both to a static orienting image and to a real-time orienting video.)

In other embodiments, as further described below with reference to FIGS. 2-5, system 10 comprises a single display, which displays both the main video and the orienting image, typically in alternation with one another.

Optionally, operator 25 may control imaging system 22 using any suitable input interface. For example, the operator may adjust the field of view (FOV) of any of the imaging devices belonging to the imaging system.

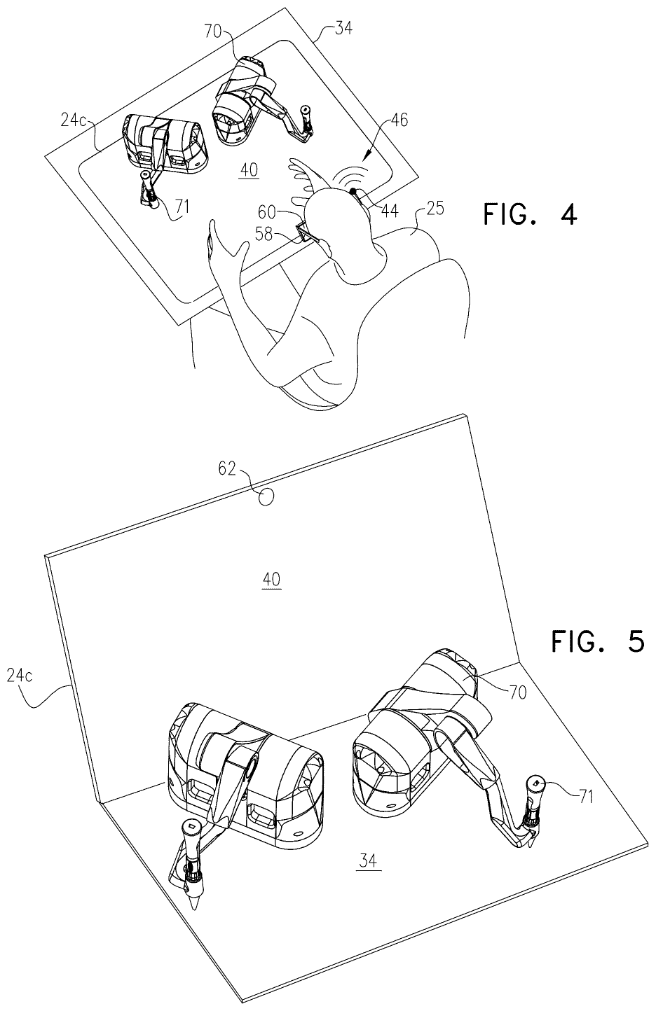

In general, processor 28 may be embodied as a single processor, or as a cooperatively networked or clustered set of processors. For example, the functionality of processor 28, as described herein, may be performed cooperatively by a first processor located in console 36 and a second processor located in device 38, a device 58 (FIG. 4), or a display 24c (FIGS. 3-5).

The functionality of processor 28 may be implemented solely in hardware, e.g., using one or more fixed-function or general-purpose integrated circuits, Application-Specific Integrated Circuits (ASICs), and/or Field-Programmable Gate Arrays (FPGAs). Alternatively, this functionality may be implemented at least partly in software. For example, processor 28 may be embodied as a programmed processor comprising, for example, a central processing unit (CPU) and/or a Graphics Processing Unit (GPU). Program code, including software programs, and/or data may be loaded for execution and processing by the CPU and/or GPU. The program code and/or data may be downloaded to the processor in electronic form, over a network, for example. Alternatively or additionally, the program code and/or data may be provided and/or stored on non-transitory tangible media, such as magnetic, optical, or electronic memory. Such program code and/or data, when provided to the processor, produce a machine or special-purpose computer, configured to perform the tasks described herein.

### Displaying the Orienting Image

Reference is now made to FIG. 2, which is a schematic illustration of an augmented-reality display of an orienting image 40, in accordance with some embodiments of the present invention. (It is noted that, notwithstanding FIG. 2, the operator typically continues to hold control tools 71 while viewing the orienting image.)

As described above with reference to FIG. 1, in some embodiments, orienting image 40 is displayed on extended-reality display 24b, such that the orienting image augments or replaces the operator's view of the real world.

Typically, in such embodiments, device 38 comprises extended-reality display 24b, along with an orientation sensor 44 configured to communicate, to the processor, a signal 46 indicating the orientation of device 38. The processor is configured to cause extended-reality display 24b to display orienting image 40 in response to the signal indicating that the operator is looking toward the vicinity of control tools 71, such as toward a surface (e.g., work surface 34) on which the control tools are disposed.

Thus, for example, while the operator looks ahead toward primary display 24a (FIG. 1), extended-reality display 24b may allow the operator to view the main video of the surgical site. For example, extended-reality display 24b may simply allow light to pass through to the operator's eyes. Alternatively, for embodiments in which primary display 24a is a 3D display, extended-reality display 24b may filter the light such that the main video appears in 3D, e.g., as described below, with reference to FIG. 3, for device 58. On the other hand, whenever the operator directs his gaze toward the vicinity of control tools 71 (e.g., toward a portion of surface 34 between the control tools and the operator), extended-reality display 24b may augment or replace the operator's view with orienting image 40.

Typically, orientation sensor 44 comprises an accelerometer, a gyroscope, and/or a magnetometer. For example, the orientation sensor may comprise an inertial measurement unit (IMU) or an inertial and magnetic measurement unit (IMMU). In some embodiments, device 38 comprises a wireless communication interface via which sensor 44 communicates with the processor, and via which the processor controls extended-reality display 24b.

Typically, given the reduced magnification of the orienting image, it is preferred that the operator refrain from manipulating the control tools while viewing the orienting image. Hence, in some embodiments, the processor is configured to lock the control tools responsively to signal 46 indicating that the operator is looking toward the vicinity of control tools 71, such that the operator cannot manipulate the control tools while viewing the orienting image. (Similarly, the processor may lock the control tools responsively to signal 46 indicating that the operator is gazing in any direction other than toward the primary display.) Subsequently, in response to signal 46 indicating that the operator is again looking toward primary display 24a, the processor unlocks the control tools.

In some embodiments, the processor is further configured to mark, in the orienting image, the FOV shown in the main video. For example, the processor may cause extended-reality display 24b to display a rectangular border 48 around the FOV. Thus, advantageously, the operator may get a better sense of the positions of the surgical tools relative to the surgical site.

Typically, each surgical tool is controllable by the operator only when the tool is within a predefined volume, referred to herein as a “control zone,” surrounding the surgical site. Hence, alternatively or additionally to marking the FOV shown in the main video, the processor may mark the control zone. For example, the processor may cause extended-reality display 24b to display a border 50 around the control zone and/or to display the control zone at a brightness different from that of its surroundings. This marking may facilitate moving the surgical tools into the control zone at the beginning of the procedure and/or moving the surgical tools from the control zone at the end of the procedure.

In some embodiments, as implied by FIG. 2, extended-reality display 24b is an augmented-reality display. Typically, in such embodiments, the surgical tools are not shown in the orienting image; rather, the augmented-reality display augments the operator's view of the control tools with a 3D image of the patient's anatomy. Optionally, the processor may augment each control tool with one or more portions of the corresponding surgical tool in the orienting image, such that it appears, to the operator, as if the operator were holding the surgical tool. For example, FIG. 2 shows a control tool 71 augmented with a virtual blade 52 corresponding to the blade of a surgical tool.

In other embodiments, extended-reality display 24b is a virtual-reality display. Typically, in such embodiments, the surgical tools are shown in the orienting image.

In some embodiments, the operator may toggle extended-reality display 24b between an augmented-reality mode, in which the display augments the operator's view of the real world, and a virtual-reality mode, in which the display replaces the operator's view of the real world.

In some embodiments, extended-reality display 24b also shows the main video of the surgical site, such that no separate primary display is required. For example, while the operator looks ahead, extended-reality display 24b may display the main video (e.g., in 3D) without augmentation, while blocking the passage of outside light to the operator's eyes. On the other hand, while the operator looks toward the vicinity of the control tools, extended-reality display 24b may display orienting image 40 as described above. Alternatively or additionally, regardless of the direction of the operator's gaze, the operator may switch between the main video and the orienting image, by instructing the processor, via any suitable input interface (e.g., a button or foot pedal), to change the display.

Reference is now made to FIG. 3, which is a schematic illustration of a surgical workspace, in accordance with some embodiments of the present invention. Reference is also made to FIG. 4, which is a schematic illustration of operator 25 using the surgical workspace shown in FIG. 3, in accordance with some embodiments of the present invention. (It is noted that, notwithstanding FIG. 4, the operator typically continues to hold control tools 71 while viewing the orienting image.) Reference is also made to FIG. 5, which is a schematic illustration of an alternate surgical workspace, in accordance with some embodiments of the present invention.

In some embodiments, the system comprises a 3D display 24c, and processor 28 causes display 24c to display orienting image 40. (The processor may communicate with display 24c over any suitable wired or wireless communication interface.) Display 24c may be mounted on, or integrated into, surface 34.

As shown in FIG. 4, to enable the appearance of image 40 in three dimensions, the system may further comprise a device 58, comprising 3D glasses for example, which is wearable over the eyes of operator 25 and comprises lenses 60 configured for viewing image 40 via the lenses. For example, display 24c may superimpose two images through different respective polarizing filters, and lenses 60 may be polarizing lenses that match, respectively, the polarization of these two images. Alternatively, display 24c and device 58 may use any other suitable technology to create the illusion of three dimensions.

Alternatively, display 24c may provide the illusion of three dimensions even without device 58. For example, as shown in FIG. 5, display 24c may use an imaging sensor 62 (e.g., an integrated imaging sensor) to track the eyes of the operator as the operator looks toward the display, and display image 40 responsively to this eye tracking. For example, display 24c may comprise a Spatial Reality Display by Sony, which uses such techniques for displaying in 3D.

In some embodiments, as shown in FIGS. 3-4, display 24c is positioned underneath control tools 71. For example, control components 70 may sit on display 24c, such that the control tools are positioned over the display. Alternatively, the control components may be mounted to a vertical surface 56 behind display 24c, such that the control tools are positioned over the display. (It is noted that display 24c may be positioned underneath control tools 71 regardless of whether the display requires device 58.)

Typically, in such embodiments, image 40 does not show the surgical tools. Rather, the operator views the control tools posed, relative to the patient, as are the actual surgical tools.

In general, display 24c may have any suitable orientation. For example, the display may be horizontal, i.e., parallel to the floor. Alternatively, as shown in FIG. 3, the display may be tilted such that the top 54 of the display, which is farther from the operator, is higher than the bottom 55 of the display, which is closer to the operator. In some embodiments, the angle θ of this tilt is 1-10 degrees; advantageously, such a tilt angle may give the appearance of standing over the patient. In other embodiments (e.g., embodiments in which display 24c comprises a Spatial Reality Display), the angle of this tilt is greater, e.g., approximately 45 degrees.

Optionally, the processor may mark, in orienting image 40, the FOV shown in the main video and/or the control zone, as described above with reference to FIG. 2.

Typically, while controlling control tools 71, the operator gazes toward primary display 24a (FIG. 1) so as to view the main video. In contrast, whenever the operator requires orientation, the operator gazes toward display 24c. As noted above, it is typically preferred that the procedure be paused while the operator is looking away from the primary display. Hence, in some embodiments, the system further comprises a sensor configured to communicate, to the processor, a signal indicating the gaze direction of the operator, and the processor is further configured to lock the control tools responsively to the signal indicating that the operator is looking toward the 3D display. (Similarly, the processor may lock the control tools responsively to the signal indicating that the operator is gazing in any direction other than toward the primary display.) Alternatively or additionally, based on the signal, the processor may cause display 24c to display image 40 only when the operator is looking toward the display.

For example, as shown in FIG. 4, device 58 may comprise orientation sensor 44, which is configured to communicate signal 46, which indicates the orientation of device 58, to the processor. (For example, device 58 may comprise a wireless communication interface via which signal 46 is communicated.) In response to the signal indicating that the operator is looking toward display 24c, the processor may lock control tools 71. Subsequently, the processor may unlock the control tools in response to the signal indicating that the operator is again looking toward the primary display.

Alternatively, for example, the sensor may comprise an imaging sensor, such as imaging sensor 62 (FIG. 5), and the signal may include an image of the operator's face (or at least the operator's eyes), which the processor may process so as to ascertain the gaze direction of the operator. The processor may thus lock the control tools responsively to the image indicating that the operator is looking toward display 24c.

Alternatively, display 24c may also display the main video, such that no separate primary display is required. As described above with reference to FIG. 2, the operator may switch between the main video and the orienting image using any suitable input interface.

Embodiments of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described with reference to the Figures, computer processor 28 typically acts as a special purpose robotic-system computer processor. Typically, the operations described herein that are performed by computer processor 28 transform the physical state of a memory, which is a real physical article, to have a different magnetic polarity, electrical charge, or the like depending on the technology of the memory that is used. For some embodiments, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A system for performing robotic surgery at a surgical site on a patient, the system comprising:
a control tool, configured for manipulation by an operator;
one or more displays; and
a processor, configured to:
cause a robotic arm, which holds a surgical tool, to manipulate the surgical tool correspondingly to the manipulation of the control tool, such that the operator controls the surgical tool via the control tool;
cause the displays to display a real-time video of the surgical site, in which the surgical site is magnified, to the operator while the operator controls the surgical tool; and
cause the displays to display at least one three-dimensional (3D) image of the surgical site, in which the surgical site is shown with less magnification relative to the video.

2. The system according to claim 1, wherein a spatial relationship between the control tool and the surgical site as shown in the 3D image corresponds to a spatial relationship between the surgical tool and the surgical site.

3. The system according to claim 1, wherein the surgical site is shown in the 3D image without any magnification.

4. The system according to claim 1, wherein a size of the surgical site as shown in the 3D image corresponds to an actual size of the surgical site.

5. The system according to claim 1,
wherein the processor is configured to scale movement of the surgical tool, relative to movement of the control tool, by a scale factor, and
wherein a size of the surgical site as shown in the 3D image is scaled, relative to an actual size of the surgical site, by an inverse of the scale factor.

6. The system according to claim 1, wherein the real-time video is 3D.

7. The system according to claim 1, wherein the processor is further configured to mark a control zone in the 3D image, the control zone being a predefined volume in which the surgical tool is controllable by the operator.

8. The system according to claim 1, wherein the at least one 3D image includes a real-time 3D video.

9. The system according to claim 1, wherein the 3D image shows the surgical tool.

10. The system according to claim 1, wherein a field of view (FOV) shown in the 3D image is larger than an FOV shown in the video.

11. The system according to claim 10, wherein the surgical site includes part of an eye of the patient, and wherein the 3D image shows a face of the patient.

12. The system according to claim 10, wherein the processor is further configured to mark, in the 3D image, the FOV shown in the video.

13. The system according to claim 1,
wherein the displays comprise a 3D display, and
wherein the processor is configured to cause the 3D display to display the 3D image.

14. The system according to claim 13, further comprising a sensor configured to communicate, to the processor, a signal indicating a gaze direction of the operator,
wherein the processor is further configured to lock the control tool responsively to the signal indicating that the operator is looking toward the 3D display.

15. The system according to claim 14, wherein the sensor comprises an imaging sensor.

16. The system according to claim 15, wherein the 3D display is configured to use the imaging sensor to track eyes of the operator as the operator looks toward the 3D display, and to display the 3D image responsively to tracking the eyes of the operator.

17. The system according to claim 14,
wherein the sensor comprises an orientation sensor, and
wherein the system further comprises a device wearable over eyes of the operator and comprising:
lenses configured for viewing the 3D image via the lenses; and
the orientation sensor.

18. The system according to claim 13, wherein the 3D display is positioned underneath the control tool.

19. The system according to claim 18, wherein the 3D display is tilted such that a top of the 3D display, which is positioned to be farther from the operator, is higher than a bottom of the 3D display, which is positioned to be closer to the operator.

20. The system according to claim 1,
wherein the displays comprise an extended-reality display, and
wherein the processor is configured to cause the extended-reality display to display the 3D image.

21. The system according to claim 20, further comprising a device wearable over eyes of the operator and comprising:
the extended-reality display; and
an orientation sensor configured to communicate, to the processor, a signal indicating an orientation of the device,
wherein the processor is configured to cause the extended-reality display to display the 3D image in response to the signal indicating that the operator is looking toward a vicinity of the control tool.

22. The system according to claim 21, wherein the processor is further configured to lock the control tool responsively to the signal indicating that the operator is looking toward the vicinity of the control tool.

23. The system according to claim 20,
wherein the extended-reality display is an augmented-reality display, and
wherein the processor is further configured to augment the control tool with one or more portions of the surgical tool in the 3D image.

24. A method for performing robotic surgery at a surgical site on a patient, the method comprising:
causing a robotic arm, which holds a surgical tool, to manipulate the surgical tool correspondingly to manipulation of a control tool by an operator, such that the operator controls the surgical tool via the control tool;
causing one or more displays to display a real-time video of the surgical site, in which the surgical site is magnified, to the operator while the operator controls the surgical tool; and
causing the displays to display at least one three-dimensional (3D) image of the surgical site, in which the surgical site is shown with less magnification relative to the video.

25. A computer software product comprising a tangible non-transitory computer-readable medium in which program instructions are stored, which instructions, when read by a processor, cause the processor to perform robotic surgery at a surgical site on a patient, by:
causing a robotic arm, which holds a surgical tool, to manipulate the surgical tool correspondingly to manipulation of a control tool by an operator, such that the operator controls the surgical tool via the control tool,
causing one or more displays to display a real-time video of the surgical site, in which the surgical site is magnified, to the operator while the operator controls the surgical tool, and
causing the displays to display at least one three-dimensional (3D) image of the surgical site, in which the surgical site is shown with less magnification relative to the video.
